(19) United States
(12) Patent Application Publication
(10) Pub. No.: US 2018/0078650 A1
(43) Pub. Date: Mar. 22, 2018

(54) COMPOSITIONS AND METHODS OF TREATING ACUTE MYELOID LEUKEMIA

(71) Applicants: Dana-Farber Cancer Institute, Inc., Boston, MA (US); Beth Israel Deaconess Medical Center, Boston, MA (US)

(72) Inventors: David AVIGAN, Sharon, MA (US); Jacalyn ROSENBLATT, Newton, MA (US); Donald KUFE, Wellesley, MA (US)

(21) Appl. No.: 15/563,151

(22) PCT Filed: Mar. 30, 2015
(86) PCT No.: PCT/US2016/024980

§ 371 Date: Sep. 29, 2017

Related U.S. Application Data
(60) Provisional application No. 62/140,325, filed on Mar. 30, 2015.

(51) Int. Cl. A61K 47/60; A61K 35/13; A61K 35/15; A61K 38/19; A61K 39/395; A61K 31/454; A61K 31/404; A61K 45/06; A61K 31/713; A61K 38/48; A61K 9/00; C12N 5/16

(52) U.S. Cl. CPC A61K 47/60; A61K 35/13; A61K 35/15; A61K 38/193; A61K 39/3955; A61K 31/454; A61K 2039/545; A61K 45/06; A61K 31/713; A61K 38/4893; A61K 9/0019; C12N 5/16; A61K 31/404

(57) ABSTRACT

The present invention provides compositions and methods for treating acute myeloid leukemia.

### RELATED APPLICATIONS

This application claims priority to, and the benefit of, U.S. Provisional Application Nos. 62/140,325, filed on Mar. 30, 2015, and 62/257,943, filed on Nov. 20, 2015, the contents of each of which are incorporated herein in their entireties.

### GOVERNMENT INTEREST

This invention was made with government support under [ ] awarded by the [ ]. The government has certain rights in the invention.

### FIELD OF THE INVENTION

The present invention relates generally to cellular immunology and more particularly to and methods for treating acute myeloid leukemia (AML).

### BACKGROUND OF THE INVENTION

Acute myelogenous leukemia is a hematological malignancy characterized by the proliferation and accumulation of immature hematopoietic cells in the bone marrow and peripheral blood. These leukemic blasts replace the bone marrow, and inhibit the growth and maturation of normal erythroid, myeloid, and megakaryocytic precursors. Presenting signs and symptoms are usually related to decreased production of normal hematopoietic cells. Untreated, AML is usually fatal within weeks to months from diagnosis. AML causes approximately 1.2% of all cancer deaths in the United States, and represents approximately 90% of adult acute leukemias.

The goal of therapy for acute myelogenous leukemia is to obtain and maintain a complete remission. Most remission induction regimens consist of a combination of an anthracycline and cytosine arabinoside. 60%-70% of patients achieve a complete remission with induction chemotherapy, 25-40% requiring more than one course to achieve a complete remission (51). Optimal treatment for patients who achieve a complete remission is controversial. Chemotherapy is often not curative for patients with AML due to the development of resistance particularly by early leukemia progenitor cells that serve as a reservoir for disease recurrence. In contrast, the potential efficacy of cellular immunotherapy in this setting is highlighted by the decreased incidence of disease relapse following allogeneic transplantation due to the anti-leukemic effects of alloreactive donor effector cells. This graft versus disease effect is manifested in patients with AML by the comparatively higher risk of relapse for patients undergoing autologous or T cell depleted transplantation and the capacity of donor lymphocytes to induce disease regression post-transplant in a subset of patients. However, the lack of specificity of the alloreactive response results in graft versus host disease, which remains a major source of morbidity and mortality following allogeneic transplantation. As a result, improved disease control associated with allogeneic transplantation is counterbalanced by increased treatment related mortality. An intensive area of research involves developing immunotherapeutic strategies to more selectively target leukemia cells, resulting in improved treatment efficacy while minimizing associated morbidity. Thus a need exists for AML specific immunotherapy.

### SUMMARY OF THE INVENTION

The invention features methods of treating acute myeloid leukemia (AML) cancer in a patient by administering to said patient in post chemotherapy induced remission or active disease a composition containing a population of autologous dendritic cell/AML tumor cell fusions (DC/AML fusions). The composition is administered 4 to 12 weeks following the completion of chemotherapy. The composition contains about 1×10⁶ to 1×10⁷ DC/AML cell fusions. Preferably, the composition contains about 5×10⁶ DC/AML cell fusions. The composition is administered at 4 week intervals.

In various aspects the method further includes administering GM-CSF. The GM-CSF is administered daily for 4 days. The GM-CSF is administered at a dose of 100 ug. The GM-CSF is administered at each dose of said DC/MM cell fusions.

In other aspects the method further includes administering to the subject a checkpoint inhibitor. The checkpoint inhibitor is administered one week after the DC/MM fusions. The checkpoint inhibitor is a PD1, PDL1, PDL2, TIM3, LAG3 inhibitor. Preferably, the checkpoint inhibitor is a PD1, PDL1, TIM3, LAG3 antibody. In other aspects the method further includes administering to the subject an agent that target regulatory T cells

In a further aspect, the method further includes administering to the subject an immunomodulatory agent. The immunomodulatory agent is lenalidomide or pomalinomide or apremilast.

In yet another aspect, he method further includes administering to the subject a TLR agonist, CPG ODN, polyIC, or tetanus toxoid.

The invention also features a method of producing a fused cell population by providing a population of AML cells and a population of dendritic cells (DC) obtained from the same subject; mixing the tumor cells and the DC at a ratio of 1:10 to 1:3 to produce a mixed cell population; adding polyethylene glycol (PEG) to the mixed cell population in an amount sufficient to mediate fusion of the tumor cell and DC cell to produce a fused cell population. Optionally, the invention further includes culturing the fused cell population in a culture media with 10% heat inactivated autologous plasma and GM-CSF and quantifying the number of cells in the fused cell population that co-express unique DC or leukemia markers.

In another aspect, a cell is provided that is produced by providing a population of AML cells and a population of dendritic cells (DC) obtained from the same subject; mixing the tumor cells and the DC at a ratio of 1:10 to 1:3 to produce a mixed cell population; adding polyethylene glycol (PEG) to the mixed cell population in an amount sufficient to mediate fusion of the tumor cell and DC cell to produce a fused cell population. In another aspect, the cell population is substantially free of endotoxin microbial contamination and mycoplasma.

Unless otherwise defined, all technical and scientific terms used herein have the same meaning as commonly understood by one of ordinary skill in the art to which this invention pertains. Although methods and materials similar or equivalent to those described herein can be used in the practice of the present invention, suitable methods and materials are described below. All publications, patent applications, patents, and other references mentioned herein are expressly incorporated by reference in their entirety. In cases of conflict, the present specification, including definitions, will control. In addition, the materials, methods, and examples described herein are illustrative only and are not intended to be limiting.

Other features and advantages of the invention will be apparent from and encompassed by the following detailed description and claims.

### BRIEF DESCRIPTION OF THE DRAWINGS

FIG. 1 is a series of histological micrographs of a biopsy taken from a vaccine site. The histological sections have been prepared counterstained with hematoxylin and eosin, or processed for immunological staining to detect CD4, CD8, or CD1a expression.

FIGS. 2A-2E are a series of graphs that depict the cellular immunologic response to vaccination. FIG. 2A depicts a series of flow cytometry graphs that quantify the amounts of CD8/INF-γ+ cells at in either pre-vac (pre-vaccination), or following 1 month, 3 months and 6 months post-vaccination. FIG. 2B is a graph that depicts the percentage of CD8/INF-γ+ cells in study participants. The solid black bars in the graph represent post-vaccination conditions. FIG. 2C is a graph that depicts the percentage of CD4/IFN-γ+ cells in study participants. The solid black bars in the graph represent post-vaccination conditions. FIGS. 2D and 2E are a series of graphs that depict pre-vaccination levels of CD4/IFN-γ+ cells (FIG. 2E) or CD8/INF-γ+ cells (FIG. 2D) to those measured one month following the last vaccine.

FIGS. 3A and 3B are a series of graphs that depict the percentage progression free survival (FIG. 3A) or the percentage overall survival over a two-year period.

FIGS. 4A-4D are a series of graphs that depict the expansion of WT-1, NY-ESO, MUC1, Survivin pentamer positive cells following vaccination. CD8+ T cells binding the MUC1 (FIG. 4C), WT-1 (FIG. 4A), Survivin (FIG. 4D) and NY-ESO (FIG. 4B) pentamers were quantified at serial time points (prior to each vaccination and at 1, 3, 6 months post vaccination) in patients who are HLA-A2.1. Binding to a control tetramer was quantified in parallel and the control value was subtracted from that obtained for the indicated tetramer. Mean values of 4 patients are presented demonstrating a marked increase in MUC1, WT1, and NY-ESO tetramer+ cells following vaccination.

### DETAILED DESCRIPTION OF THE INVENTION

The invention features immune system-stimulating compositions that contain cells formed by fusion between autologous dendritic cells (DCs) and tumor cells. Specifically, the invention provides cell fusion of autologous DCs and acute myelogenous leukemia (AML) cells obtained from a subject that has AML. Also provide are methods of treating AML by administering to a patient whom is in a post chemotherapy induced remission the autologous cell fusions according to the invention.

AML offers a unique therapeutic challenge. A majority of patients achieve a remission following chemotherapy; however, only a small minority experience durable responses particularly in patients over the age of 60 in which only 15-20% of patients remain free of leukemia at two years. The high rate of recurrence following induction chemotherapy is thought to arise from the persistence of clonal populations intrinsically resistant to cytotoxic injury, including the malignant stem cell population resulting in the repopulation of disease within 6-12 months. In contrast, the unique efficacy of cellular immunotherapy for AML is highlighted by the observation that allogeneic transplantation is potentially curative for a subset of patients due to the immunologic capacity of alloreactive lymphocytes to broadly eradicate the malignant clonal population. However, the application of this strategy is limited by often prohibitive treatment associated morbidity and mortality due to targeting of normal tissues in the context of graft versus host disease.

An important focus of investigation is therefore the development of AML vaccines to induce immune responses that will more selectively eliminate AML cells. However, to our knowledge, there are presently no vaccines that have shown promise in the treatment of AML. Strategies to design AML vaccines have included single antigen based approaches such as WT1 peptide administered with adjuvant 16,17, dendritic cells loaded with tumor associated antigens, or the use of AML cells differentiated into DCs. Immune responses have been observed in several studies; however, there has been limited evidence for clinical efficacy. Of note, dramatic responses have been seen in a subset of patients with acute lymphocytic leukemia undergoing adoptive immunotherapy with chimeric antigen receptor T cell therapy in which persistence of the tumor specific T cells has been associated with durable response. However, in a majority of studies the presence of circulating CAR-T cells is largely extinguished beyond 2 months raising the potential concern for late relapse.

The clinical effects of the vaccine were observed in the context of sustained induction of AML specific immunity as measured by the expansion of AML specific CD4 and CD8 T cells in the peripheral blood. A concomitant rise in leukemia specific T cells in the bone marrow further supports the notion that the immune response is generated in this critical microenvironment. The specificity of the immune response is further supported by the expansion of T cells recognizing leukemia associated antigens, WT1, MUC1, and NYESO. In this context, MUC1 is uniquely expressed by leukemia stem cells, as compared to normal hematopoietic stem cells, consistent with the potential targeting of this self-renewing population. The expansion of AML specific T cells peaked at 2 months after vaccination and persisted at 6 months following treatment, the last time point measured. Thus the role of booster vaccination in maintaining response should be evaluated.

In one embodiment, a personalized vaccine is described in which patient derived AML cells are fused with autologous dendritic cells (DCs), incorporating antigens that capture the heterogeneity of the leukemia cell population and presenting them in the context of the potent antigen presenting machinery of the DC. Additionally, DC/AML fusions induce a polyclonal helper and cytotoxic T cell immune response that includes targeting of the leukemia stem cell population. Without being bound to any specific mechanism, theory or hypothesis, one hypothesis can be that chemotherapy induced remission would augment vaccine response by optimizing tumor cytoreduction and reducing the immunosuppressive effect on the bone marrow microenvironment and create an ideal platform to immunologically target persisting chemotherapy resistant clones.

DCs can be obtained from bone marrow cultures, peripheral blood, spleen, or any other appropriate tissue of a mammal using protocols known in the art. Bone marrow contains DC progenitors, which, upon treatment with cytokines, such as granulocyte-macrophage colony-stimulating factor (“GM-CSF”) and interleukin 4 (“IL-4”), proliferate and differentiate into DCs. Tumor necrosis cell factor (TNF) is optionally in conjunction with GM-CSF and/or IL-4 to promote maturation of DCs. DCs obtained from bone marrow are relatively immature (as compared to, for instance, spleen DCs). GM-CSF/IL-4 stimulated DC express MHC class I and class II molecules, B7-1, B7-2, ICAM, CD40 and variable levels of CD83. These immature DCs are more amenable to fusion (or antigen uptake) than the more mature DCs found in spleen, whereas more mature DCs are relatively more effective antigen presenting cells. Peripheral blood also contains relatively immature DCs or DC progenitors, which can propagate and differentiate in the presence of appropriate cytokines such as GM-CSF and-which can also be used in fusion.

Preferably, the DCs are obtained from peripheral blood. For example, the DCs are obtained from the patients peripheral blood after it has been documented that the patient is in complete remission.

The DCs must have sufficient viability prior to fusion. The viability of the DCs is at least 70%, at least 75%, at least 80% or greater.

Prior to fusion the population of the DCs are free of components used during the production, e.g., cell culture components and substantially free of mycoplasm, endotoxin, and microbial contamination. Preferably, the population of DCs has less than 10, 5, 3, 2, or 1 CFU/swab. Most preferably the population of DCs has 0 CFU/swab.

The tumor cells used in the invention are acute myelogenous leukemia cells. The acute myelogenous leukemia cells are obtained from a patient having acute myelogenous leukemia. In preferred embodiments, the patient has are newly diagnosed with AML or in their first relapse.

The tumor cells must have sufficient viability prior to fusion. The viability of the tumor cells is at least 50%, at least 60%, at least 70%, at least 80% or greater.

Prior to fusion the population of tumor cells are free of components used during the production, e.g., cell culture components and substantially free of mycoplasm, endotoxin, and microbial contamination. Preferably, the population of tumor cell population has less than 10, 5, 3, 2, or 1 CFU/swab. Most preferably the population of tumor cells has 0 CFU/swab. The endotoxin level in the population of tumor cells is less than 20 EU/mL, less than 10 EU/mL or less than 5 EU/mL.

If the tumor cells die or at least fail to proliferate in the presence of a given reagent and this sensitivity can be overcome by the fusion with DCs, the post-fusion cell mixtures containing the fused as well as the parental cells may optionally be incubated in a medium containing this reagent for a period of time sufficient to eliminate most of the unfused cells. The fusion product is used directly after the fusion process (e.g., in antigen discovery screening methods or in therapeutic methods) or after a short culture period.

Fused cells are irradiated prior to clinical use.

In the event that the fused cells lose certain DC characteristics such as expression of the APC-specific T-cell stimulating molecules, primary fused cells can be refused with dendritic cells to restore the DC phenotype. The refused cells (i.e., secondary fused cells) are found to be highly potent APCs. The fused cells can be refused with the dendritic or non-dendritic parental cells as many times as desired.

Fused cells that express MHC class II molecules, B7, or other desired T-cell stimulating molecules can also be selected by panning or fluorescence-activated cell sorting with antibodies against these molecules.

Fusion between the DCs and the tumor cells can be carried out with well-known methods such as those using polyethylene glycol (“PEG”), Sendai virus, or electrofusion. DCs are autologous or allogeneic. (See, e.g., U.S. Pat. No. 6,653,848, which is herein incorporated by reference in its entirety). The ratio of DCs to tumor cells in fusion can vary from 1:100 to 1000:1, with a ratio higher than 1:1 being preferred. Preferably, the ratio is 1:1, 5:1, or 10:1. Most preferably, the ratio of DCs to tumor cells is 10:1 or 3:1. After fusion, unfused DCs usually die off in a few days in culture, and the fused cells can be separated from the unfused parental non-dendritic cells by the following two methods, both of which yield fused cells of approximately 50% or higher purity, i.e., the fused cell preparations contain less than 50%, and often less than 30%, unfused cells.

Specifically, one method of separating unfused cells from fused cells is based on the different adherence properties between the fused cells and the non-dendritic parental cells. It has been found that the fused cells are generally lightly adherent to tissue culture containers. Thus, if the non-dendritic parental cells are much more adherent, e.g., in the case of carcinoma cells, the post-fusion cell mixtures can be cultured in an appropriate medium for a short period of time (e.g., 5-10 days). Subsequently, the fused cells can be gently dislodged and aspirated off, while the unfused cells grow firmly attached to the tissue culture containers. Conversely, if the tumor cells grow in suspension, after the culture period, they can be gently aspirated off while leaving the fused cells loosely attached to the containers. Alternatively, the hybrids are used directly without an in vitro cell culturing step.

Fused cells obtained by the above-described methods typically retain the phenotypic characteristics of DCs. For instance, these fused cells express T-cell stimulating molecules such as MHC class II protein, B7-1, B7-2, and adhesion molecules characteristic of APCs such as ICAM-1. The fused cells also continue to express cell-surface antigens of the tumor cells such as MUC-1, CD34, CD117, and CD38 are therefore useful for inducing immunity against the cell-surface antigens.

In the event that the fused cells lose certain DC characteristics such as expression of the APC-specific T-cell stimulating molecules, they (i.e., primary fused cells) can be re-fused with dendritic cells to restore the DC phenotype. The re-fused cells (i.e., secondary fused cells) are found to be highly potent APCs, and in some cases, have even less tumorigenicity than primary fused cells. The fused cells can be re-fused with the dendritic or non-dendritic parental cells as many times as desired.

The phenotypic characteristics of DC/AML fusions are examined. Specifically, fusion of DCs/AML fusions co-express: a) CD86 and CD117 or CD 34 or MUC-1 orb) CD83 and CD117 or CD34 or MUC-1; c) if the leukemia cells do not express DR, then DR and CD117 or CD34 or MUC-1.

The fused cells may be frozen before administration. The fused cells are frozen in a solution containing 10% DMSO in 90% autologous heat inactivated autologous plasma.

The fused cells of the invention can be used to stimulate the immune system of a mammal for treatment or prophylaxis of acute myeloid leukemia. For instance, to treat a acute myeloid leukemia in a human, a composition containing fused cells formed by his own DCs and tumor cells can be administered to him, e.g., at a site near the lymphoid tissue. In some embodiments the subject is in post chemotherapy induced remission. Preferably the fused cells are administered 4 to 12 weeks following the completion of chemotherapy. More preferably, the fused cells are administered to the subject within 4 weeks of hematopoietic recovery following the chemotherapy=Alternatively, the fused cells are administered during the early period of hematopoietic recovery in which levels of circulating and bone marrow regulatory T cells are at a minimum or in combination with agents the target regulatory T cells. Another criteria for determining the timing of the administration of the fused cell is at a time post-transplant in which there is expansion of AML specific T cells post-chemotherapy as measured by the percentage of CD4 and/or CD8 T cells that express IFNγ in response to ex vivo exposure to autologous tumor lysate or the percentage of T cells that bind to tetramers or pentamers expressing AML specific antigens such as WT1, Survivin, NY-ESO, MUC1, and PRAME.

Preferably, the vaccine is administered to four different sites near lymphoid tissue. The composition may be given multiple times (e.g., two to five, preferably three) at an appropriate intervals, preferably, four weeks and dosage (e.g., approximately 10⁵-10⁸, e.g., about 0.5×10⁶ to 1×10⁶, fused cells per administration). Preferably each dosage contains approximately 1×10⁶ to 1×10⁷ fused cells. More preferably each dosage contains approximately 5×10⁶ fused cells. In addition the fused cells the patient further receives GM-CSF. The GM-CSF is administered on the day the fused cells are administered and for daily for three subsequent days. The GM-CSF is administered subcutaneously at a dose of 100 ug. The GM-CSF is administered at the site where the fused cells are administered.

Optionally, the patient further receives a checkpoint inhibitor. The check point inhibitor is administered contemporaneously with the fused cell, prior to administration of the fused cells or after administration of the fused cells. For example, the checkpoint inhibitor is administered 1 week prior to the fused cells. Preferably, the checkpoint inhibitor is administered 1 week after the fused cells. The checkpoint inhibitor is administered at 1, 2, 3, 4, 5, 6 week intervals.

By checkpoint inhibitor it is meant that at the compound inhibits a protein in the checkpoint signally pathway. Proteins in the checkpoint signally pathway include for example, PD-1, PD-L1, PD-L2, TIM3, LAG3, and CTLA-4. Checkpoint inhibitor are known in the art. For example, the checkpoint inhibitor can be a small molecule. A “small molecule” as used herein, is meant to refer to a composition that has a molecular weight in the range of less than about 5 kD to 50 daltons, for example less than about 4 kD, less than about 3.5 kD, less than about 3 kD, less than about 2.5 kD, less than about 2 kD, less than about 1.5 kD, less than about 1 kD, less than 750 daltons, less than 500 daltons, less than about 450 daltons, less than about 400 daltons, less than about 350 daltons, less than 300 daltons, less than 250 daltons, less than about 200 daltons, less than about 150 daltons, less than about 100 daltons. Small molecules can be, e.g., nucleic acids, peptides, polypeptides, peptidomimetics, carbohydrates, lipids or other organic or inorganic molecules.

Alternatively the checkpoint inhibitor is an antibody is an antibody or fragment thereof. For example, the antibody or fragment thereof is specific to a protein in the checkpoint signaling pathway, such as PD-1, PD-L1, PD-L2, TIM3, LAG3, or CTLA-4. Preferably, the checkpoint inhibitor is an antibody specific for PD-1, PD-L1, PD-L2, TIM3, LAG3, or CTLA-4.

Optionally, the patient may receive concurrent treatment with an immunomodulatory agent. These agents include lenalidomide, pomalinomide or apremilast. Lenalidomide has been shown to boost response to vaccination targeting infectious diseases and in pre-clinical studies enhances T cell response to the fusion vaccine.

Optionally the patient may undergo vaccination in combination with strategies to reduce levels of regulatory T cells. These strategies may include combining vaccination with chemotherapy, during the period of lymphopoietic reconstitution following autologous or allogeneic transplantation, and with antibodies or drugs that target regulatory T cells.

To monitor the effect of vaccination, cytotoxic T lymphocytes obtained from the treated individual can be tested for their potency against cancer cells in cytotoxic assays. Multiple boosts may be needed to enhance the potency of the cytotoxic T lymphocytes.

Compositions containing the appropriate fused cells are administered to an individual (e.g., a human) in a regimen determined as appropriate by a person skilled in the art. For example, the composition may be given multiple times (e.g., three to five times, preferably three) at an appropriate interval (e.g., every four weeks) and dosage (e.g., approximately 10⁵-10⁸, preferably about 1×10⁶ to 1×10⁷, more preferably 5×10⁶ fused cells per administration).

The composition of fused cells prior to administration to the patient must have sufficient viability. The viability of the fused cells at the time of administration is at least 50%, at least 60%, at least 70%, at least 80% or greater.

Prior to administration, the population of fused cells are free of components used during the production, e.g., cell culture components and substantially free of mycoplasm, endotoxin, and microbial contamination. Preferably, the population of fused cells has less than 10, 5, 3, 2, or 1 CFU/swab. Most preferably the population of tumor cells has 0 CFU/swab. For example, the results of the sterility testing is “negative” or “no growth”. The endotoxin level in the population of tumor cells is less than 20 EU/mL, less than 10 EU/mL or less than 5 EU/mL. The results of the myoplasm testing is “negative”.

Prior to administration, the fused cell must express at least 40%, at least 50%, at least60% CD86 as determined by immunological staining. Preferably the fused cells express at least 50% CD86. The fused cells may also express MUC1, CD34, CD117, or CD38.

More specifically, all final cell product must conform with rigid requirements imposed by the Federal Drug Administration (FDA). The FDA requires that all final cell products must minimize “extraneous” proteins known to be capable of producing allergenic effects in human subjects as well as minimize contamination risks. Moreover, the FDA expects a minimum cell viability of 70%, and any process should consistently exceed this minimum requirement.

Definitions

The practice of the present invention employs, unless otherwise indicated, conventional techniques of molecular biology, microbiology, cell biology and recombinant DNA, which are within the skill of the art. See, e.g., Sambrook, Fritsch and Maniatis, MOLECULAR CLONING: A LABORATORY MANUAL, 2^{nd} edition (1989); CURRENT PROTOCOLS IN MOLECULAR BIOLOGY (F. M. Ausubel et al. eds., (1987)); the series METHODS IN ENZYMOLOGY (Academic Press, Inc.): PCR 2: A PRACTICAL APPROACH (Mi. MacPherson, B. D. Hames and G. R. Taylor eds. (1995)) and ANIMAL CELL CULTURE (Rd. Freshney, ed. (1987)).

As used herein, certain terms have the following defined meanings. As used in the specification and claims, the singular form “a”, “an” and “the” include plural references unless the context clearly dictates otherwise. For example, the term “a cell” includes a plurality of cells, including mixtures thereof.

The term “immune effector cells” refers to cells that specifically recognize an antigen present, for example on a neoplastic or tumor cell. For the purposes of this invention, immune effector cells include, but are not limited to, B cells; monocytes; macrophages; NK cells; and T cells such as cytotoxic T lymphocytes (CTLs), for example CTL lines, CTL clones, and CTLs from tumor, inflammatory sites or other infiltrates. “T-lymphocytes” denotes lymphocytes that are phenotypically CD3+, typically detected using an anti-CD3 monoclonal antibody in combination with a suitable labeling technique. The T-lymphocytes of this invention are also generally positive for CD4, CD8, or both. The term “naïve” immune effector cells refers to immune effector cells that have not encountered antigen and is intended to by synonymous with unprimed and virgin. “Educated” refers to immune effector cells that have interacted with an antigen such that they differentiate into an antigen-specific cell.

The terms “antigen presenting cells” or “APCs” includes both intact, whole cells as well as other molecules which are capable of inducing the presentation of one or more antigens, preferably with class I MHC molecules. Examples of suitable APCs are discussed in detail below and include, but are not limited to, whole cells such as macrophages, dendritic cells, B cells; purified MHC class I molecules complexed to β2-microglobulin; and foster antigen presenting cells.

Dendritic cells (DCs) are potent APCs. DCs are minor constituents of various immune organs such as spleen, thymus, lymph node, epidermis, and peripheral blood. For instance, DCs represent merely about 1% of crude spleen (see Steinman et al. (1979) J. Exp. Med 149: 1) or epidermal cell suspensions (see Schuler et al. (1985) J. Exp. Med 161:526; Romani et al. J. Invest. Dermatol (1989) 93: 600) and 0.1-1% of mononuclear cells in peripheral blood (see Freudenthal et al. Proc. Natl Acad Sci USA (1990) 87: 7698). Methods for isolating DCs from peripheral blood or bone marrow progenitors are known in the art. (See Inaba et al. (1992) J. Exp. Med 175:1157; Inaba et al. (1992) J. Exp, Med 176: 1693-1702; Romani et al. (1994) J. Exp. Med. 180: 83-93; Sallusto et al. (1994) J. Exp. Med 179: 1109-1118)). Preferred methods for isolation and culturing of DCs are described in Bender et al. (1996) J. Immun. Meth. 196:121-135 and Romani et al. (1996) J. Immun. Meth 196:137-151.

Dendritic cells (DCs) represent a complex network of antigen presenting cells that are primarily responsible for initiation of primary immunity and the modulation of immune response. (See Avigan, Blood Rev. 13:51-64 (1999); Banchereau et al., Nature 392:245-52 (1998)). Partially mature DCs are located at sites of antigen capture, excel at the internalization and processing of exogenous antigens but are poor stimulators of T cell responses. Presentation of antigen by immature DCs may induce T cell tolerance. (See Dhodapkar et al., J Exp Med. 193:233-38 (2001)). Upon activation, DCs undergo maturation characterized by the increased expression of costimulatory molecules and CCR7, the chemokine receptor which promotes migration to sites of T cell traffic in the draining lymph nodes. Tumor or cancer cells inhibit DC development through the secretion of IL-10, TGF-β, and VEGF resulting in the accumulation of immature DCs in the tumor bed that potentially suppress anti-tumor responses. (See Allavena et al., Eur. J. Immunol. 28:359-69 (1998); Gabrilovich et al., Clin Cancer Res. 3:483-90 (1997); Gabrilovich et al., Blood 92:4150-66 (1998); Gabrilovich, Nat Rev Immunol 4:941-52 (2004)). Conversely, activated DCs can be generated by cytokine mediated differentiation of DC progenitors ex vivo. DC maturation and function can be further enhanced by exposure to the toll like receptor 9 agonist, CPG ODN. Moreover, DCs can be manipulated to present tumor antigens potently stimulate anti-tumor immunity. (See Asavaroenhchai et al., Proc Natl Acad Sci USA 99:931-36 (2002); Ashley et al., J Exp Med 186:1177-82 (1997)).

“Foster antigen presenting cells” refers to any modified or naturally occurring cells (wild-type or mutant) with antigen presenting capability that are utilized in lieu of antigen presenting cells (“APC”) that normally contact the immune effector cells they are to react with. In other words, they are any functional APCs that T cells would not normally encounter in vivo.

It has been shown that DCs provide all the signals required for T cell activation and proliferation. These signals can be categorized into two types. The first type, which gives specificity to the immune response, is mediated through interaction between the T-cell receptor/CD3 (“TCR/CD3”) complex and an antigenic peptide presented by a major histocompatibility complex (“MHC”) class I or II protein on the surface of APCs. This interaction is necessary, but not sufficient, for T cell activation to occur. In fact, without the second type of signals, the first type of signals can result in T cell anergy. The second type of signals, called costimulatory signals, are neither antigen-specific nor MHC restricted, and can lead to a full proliferation response of T cells and induction of T cell effector functions in the presence of the first type of signals.

Thus, the term “cytokine” refers to any of the numerous factors that exert a variety of effects on cells, for example, inducing growth or proliferation. Non-limiting examples of cytokines include, IL-2, stem cell factor (SCF), IL-3, IL-6, IL-7, IL-12, IL-15, G-CSF, GM-CSF, IL-1 α, IL-1 β, MIP-1 α, LIF, c-kit ligand, TPO, and flt3 ligand. Cytokines are commercially available from several vendors such as, for example, Genzyme Corp. (Framingham, Mass.), Genentech (South San Francisco, Calif.), Amgen (Thousand Oaks, Calif.) and Immunex (Seattle, Wash.). It is intended, although not always explicitly stated, that molecules having similar biological activity as wild-type or purified cytokines (e.g., recombinantly produced cytokines) are intended to be used within the spirit and scope of the invention and therefore are substitutes for wild-type or purified cytokines.

“Costimulatory molecules” are involved in the interaction between receptor-ligand pairs expressed on the surface of antigen presenting cells and T cells. One exemplary receptor-ligand pair is the B7 co-stimulatory molecules on the surface of DCs and its counter-receptor CD28 or CTLA-4 on T cells. (See Freeman et al. (1993) Science 262:909-911; Young et al. (1992) J. Clin. Invest 90: 229; Nabavi et al. Nature 360:266)). Other important costimulatory molecules include, for example, CD40, CD54, CD80, and CD86. These are commercially available from vendors identified above.

A “hybrid” cell refers to a cell having both antigen presenting capability and also expresses one or more specific antigens. In one embodiment, these hybrid cells are formed by fusing, in vitro, APCs with cells that are known to express the one or more antigens of interest. As used herein, the term “hybrid” cell and “fusion” cell are used interchangeably.

A “control” cell refers to a cell that does not express the same antigens as the population of antigen-expressing cells.

The term “culturing” refers to the in vitro propagation of cells or organisms on or in media of various kinds, it is understood that the descendants 30 of a cell grown in culture may not be completely identical (i.e., morphologically, genetically, or phenotypically) to the parent cell. By “expanded” is meant any proliferation or division of cells.

An “effective amount” is an amount sufficient to effect beneficial or desired results. An effective amount can be administered in one or more administrations, applications or dosages. For purposes of this invention, an effective amount of hybrid cells is that amount which promotes expansion of the antigenic-specific immune effector cells, e.g., T cells.

An “isolated” population of cells is “substantially free” of cells and materials with which it is associated in nature. By “substantially free” or “substantially pure” is meant at least 50% of the population is the desired cell type, preferably at least 70%, more preferably at least 80%, and even more preferably at least 90%. An “enriched” population of cells is at least 5% fused cells. Preferably, the enriched population contains at least 10%, more preferably at least 20%, and most preferably at least 25% fused cells.

The term “autogeneic”, or “autologous”, as used herein, indicates the origin of a cell. Thus, a cell being administered to an individual (the “recipient”) is autogeneic if the cell was derived from that individual (the “donor”) or a genetically identical individual (i.e., an identical twin of the individual). An autogeneic cell can also be a progeny of an autogeneic cell. The term also indicates that cells of different cell types are derived from the same donor or genetically identical donors. Thus, an effector cell and an antigen presenting cell are said to be autogeneic if they were derived from the same donor or from an individual genetically identical to the donor, or if they are progeny of cells derived from the same donor or from an individual genetically identical to the donor.

Similarly, the term “allogeneic”, as used herein, indicates the origin of a cell. Thus, a cell being administered to an individual (the “recipient”) is allogeneic if the cell was derived from an individual not genetically identical to the recipient. In particular, the term relates to non-identity in expressed MHC molecules. An allogeneic cell can also be a progeny of an allogeneic cell. The term also indicates that cells of different cell types are derived from genetically nonidentical donors, or if they are progeny of cells derived from genetically non-identical donors. For example, an APC is said to be allogeneic to an effector cell if they are derived from genetically non-identical donors.

A “subject” is a vertebrate, preferably a mammal, more preferably a human. Mammals include, but are not limited to, murines, simians, humans, farm animals, sport animals, and pets.

As used herein, “genetic modification” refers to any addition, deletion or disruption to a cell's endogenous nucleotides.

A “viral vector” is defined as a recombinantly produced virus or viral particle that comprises a polynucleotide to be delivered into a host cell, either in vivo, ex vivo or in vitro. Examples of viral vectors include retroviral vectors, adenovirus vectors, adeno-associated virus vectors and the like. In aspects where gene transfer is mediated by a retroviral vector, a vector construct refers to the polynucleotide comprising the retroviral genome or part thereof, and a therapeutic gene.

As used herein, the terms “retroviral mediated gene transfer” or “retroviral transduction” carries the same meaning and refers to the process by which a gene or a nucleic acid sequence is stably transferred into the host cell by virtue of the virus entering the cell and integrating its genome into the host cell genome. The virus can enter the host cell via its normal mechanism of infection or be modified such that it binds to a different host cell surface receptor or ligand to enter the cell.

Retroviruses carry their genetic information in the form of RNA. However, once the virus infects a cell, the RNA is reverse-transcribed into the DNA form that integrates into the genomic DNA of the infected cell. The integrated DNA form is called a provirus.

In aspects where gene transfer is mediated by a DNA viral vector, such as a adenovirus (Ad) or adeno-associated virus (AAV), a vector construct refers to the polynucleotide comprising the viral genome or part thereof, and a therapeutic gene. Adenoviruses (Ads) are a relatively well characterized, homogenous group of viruses, including over 50 serotypes. (See, e.g., WO 95/27071). Ads are easy to grow and do not integrate into the host cell genome. Recombinant Ad-derived vectors, particularly those that reduce the potential for recombination and generation of wild-type virus, have also been constructed. (See, WO 95/00655; WO 95/11984). Wild-type AAV has high infectivity and specificity integrating into the host cells genome. (See Hermonat and Muzyczka (1984) PNAS USA 81:6466-6470; Lebkowski et al., (1988) Mol Cell Biol 8:3988-3996).

Vectors that contain both a promoter and a cloning site into which a polynucleotide can be operatively linked are well known in the art. Such vectors are capable of transcribing RNA in vitro or in vivo, and are commercially available from sources such as Stratagene (La Jolla, Calif.) and Promega Biotech (Madison, Wis.). In order to optimize expression and/or in vitro transcription, it may be necessary to remove, add or alter 5′ and/or 3′ untranslated portions of the clones to eliminate extra, potential inappropriate alternative translation initiation codons or other sequences that may interfere with or reduce expression, either at the level of transcription or translation. Alternatively, consensus ribosome binding sites can be inserted immediately 5′ of the start codon to enhance expression. Examples of suitable vectors are viruses, such as baculovirus and retrovirus, bacteriophage, cosmid, plasmid, fungal vectors and other recombination vehicles typically used in the art which have been described for expression in a variety of eucaryotie and prokaryotic hosts, and may be used for gene therapy as well as for simple protein expression.

Among these are several non-viral vectors, including DNA/liposome complexes, and targeted viral protein DNA complexes. To enhance delivery to a cell, the nucleic acid or proteins of this invention can be conjugated to antibodies or binding fragments thereof which bind cell surface antigens, e.g., TCR, CD3 or CD4. Liposomes that also comprise a targeting antibody or fragment thereof can be used in the methods of this invention. This invention also provides the targeting complexes for use in the methods disclosed herein.

Polynucleotides are inserted into vector genomes using methods well known in the art. For example, insert and vector DNA can be contacted, under suitable conditions, with a restriction enzyme to create complementary ends on each molecule that can pair with each other and be joined together with a ligase. Alternatively, synthetic nucleic acid linkers can be ligated to the termini of restricted polynucleotide. These synthetic linkers contain nucleic acid sequences that correspond to a particular restriction site in the vector DNA. Additionally, an oligonucleotide containing a termination codon and an appropriate restriction site can be ligated for insertion into a vector containing, for example, some or all of the following: a selectable marker gene, such as the neomycin gene for selection of stable or transient transfectants in mammalian cells; enhancer/promoter sequences from the immediate early gene of human CMV for high levels of transcription; transcription termination and RNA processing signals from SV4O for mRNA stability; SV40 polyoma origins of replication and ColEI for proper episomal replication; versatile multiple cloning sites; and T7 and SP6 RNA promoters for in vitro transcription of sense and antisense RNA. Other means are well known and available in the art.

As used herein, “expression” refers to the process by which polynucleotides are transcribed into mRNA and translated into peptides, polypeptides, or proteins. If the polynucleotide is derived from genomic DNA, expression may include splicing of the mRNA, if an appropriate eukaryotic host is selected. Regulatory elements required for expression include promoter sequences to bind RNA polymerase and transcription initiation sequences for ribosome binding. For example, a bacterial expression vector includes a promoter such as the lac promoter and for transcription initiation the Shine-Dalgarno sequence and the start codon AUG (Sambrook et al. (1989), supra). Similarly, a eukaryotic expression vector includes a heterologous or homologous promoter for RNA polymerase II, a downstream polyadenylation signal, the start codon AUG, and a termination codon for detachment of the ribosome. Such vectors can be obtained commercially or assembled by the sequences described in methods well known in the art, for example, the methods described above for constructing vectors in general.

The terms “major histocompatibility complex” or “MHC” refers to a complex of genes encoding cell-surface molecules that are required for antigen presentation to immune effector cells such as T cells and for rapid graft rejection. In humans, the MHC complex is also known as the HLA complex. The proteins encoded by the MHC complex are known as “MHC molecules” and are classified into class I and class II MHC molecules. Class I MHC molecules include membrane heterodimeric proteins made up of an α chain encoded in the MHC associated noncovalently with β2-microglobulin. Class I MHC molecules are expressed by nearly all nucleated cells and have been shown to function in antigen presentation to CD8+T cells. Class I molecules include HLA-A, -B, and -C in humans. Class II MHC molecules also include membrane heterodimeric proteins consisting of noncovalently associated and J3 chains. Class II MHCs are known to function in CD4+ T cells and, in humans, include HLA-DP, -DQ, and DR. The term “MHC restriction” refers to a characteristic of T cells that permits them to recognize antigen only after it is processed and the resulting antigenic peptides are displayed in association with either a class I or class II MHC molecule. Methods of identifying and comparing MHC are well known in the art and are described in Allen M. et al. (1994) Human Imm. 40:25-32; Santamaria P. et al. (1993) Human Imm. 37:39-50; and Hurley C. K. et al. (1997) Tissue Antigens 50:401-415.

The term “sequence motif” refers to a pattern present in a group of 15 molecules (e.g., amino acids or nucleotides). For instance, in one embodiment, the present invention provides for identification of a sequence motif among peptides present in an antigen. In this embodiment, a typical pattern may be identified by characteristic amino acid residues, such as hydrophobic, hydrophilic, basic, acidic, and the like.

The term “peptide” is used in its broadest sense to refer to a compound of two or more subunit amino acids, amino acid analogs, or peptidomimetics. The subunits may be linked by peptide bonds. In another embodiment, the subunit may be linked by other bonds, e.g. ester, ether, etc.

As used herein the term “amino acid” refers to either natural and/or 25 unnatural or synthetic amino acids, including glycine and both the D or L optical isomers, and amino acid analogs and peptidomimetics. A peptide of three or more amino acids is commonly called an oligopeptide if the peptide chain is short. If the peptide chain is long, the peptide is commonly called a polypeptide or a protein.

As used herein, “solid phase support” is used as an example of a “carrier” and is not limited to a specific type of support. Rather a large number of supports are available and are known to one of ordinary skill in the art. Solid phase supports include silica gels, resins, derivatized plastic films, glass beads, cotton, plastic beads, alumina gels. A suitable solid phase support may be selected on the basis of desired end use and suitability for various synthetic protocols. For example, for peptide synthesis, solid phase support may refer to resins such as polystyrene (e.g., PAM-resin obtained from Bachem Inc., Peninsula Laboratories, etc.), POLYHIPE® resin (obtained from Aminotech, Canada), polyamide resin (obtained from Peninsula Laboratories), polystyrene resin grafted with polyethylene glycol (TentaGel®, Rapp Polymere, Tubingen, Germany) or polydimethylacrylamide resin (obtained from MilligenlBiosearch, California). In a preferred embodiment for peptide synthesis, solid phase support refers to polydimethylacrylamide resin.

The term “aberrantly expressed” refers to polynucleotide sequences in a cell or tissue which are differentially expressed (either over-expressed or under-expressed) when compared to a different cell or tissue whether or not of the same tissue type, i.e., lung tissue versus lung cancer tissue.

“Host cell” or “recipient cell” is intended to include any individual cell or cell culture which can be or have been recipients for vectors or the incorporation of exogenous nucleic acid molecules, polynucleotides and/or proteins. It also is intended to include progeny of a single cell, and the progeny may not necessarily be completely identical (in morphology or in genomic or total DNA complement) to the original parent cell due to natural, accidental, or deliberate mutation. The cells may be prokaryotic or eukaryotic, and include but are not limited to bacterial cells, yeast cells, animal cells, and mammalian cells, e.g., murine, rat, simian or human.

An “antibody” is an immunoglobulin molecule capable of binding an antigen. As used herein, the term encompasses not only intact immunoglobulin molecules, but also anti-idiotypic antibodies, mutants, fragments, fusion proteins, humanized proteins and modifications of the immunoglobulin molecule that comprise an antigen recognition site of the required specificity.

An “antibody complex” is the combination of antibody and its binding partner or ligand.

A “native antigen” is a polypeptide, protein or a fragment containing an epitope, which induces an immune response in the subject.

The term “isolated” means separated from constituents, cellular and otherwise, in which the polynucleotide, peptide, polypeptide, protein, antibody, or fragments thereof, are normally associated with in nature. As is apparent to those of skill in the art, a non-naturally occurring polynucleotide, peptide, polypeptide, protein, antibody, or fragments thereof, does not require “isolation” to distinguish it from its naturally occurring counterpart. In addition, a “concentrated”, “separated” or “diluted” polynucleotide, peptide, polypeptide, protein, antibody, or fragments thereof, is distinguishable from its naturally occurring counterpart in that the concentration or number of molecules per volume is greater than “concentrated” or less than “separated” than that of its naturally occurring counterpart. A polynucleotide, peptide, polypeptide, protein, antibody, or fragments thereof, which differs from the naturally occurring counterpart in its primary sequence or for example, by its glycosylation pattern, need not be present in its isolated form since it is distinguishable from its naturally occurring counterpart by its primary sequence, or alternatively, by another characteristic such as glycosylation pattern. Although not explicitly stated for each of the inventions disclosed herein, it is to be understood that all of the above embodiments for each of the compositions disclosed below and under the appropriate conditions, are provided by this invention. Thus, a non-naturally occurring polynucleotide is provided as a separate embodiment from the isolated naturally occurring polynucleotide. A protein produced in a bacterial cell is provided as a separate embodiment from the naturally occurring protein isolated from a eukaryotic cell in which it is produced in nature.

A “composition” is intended to mean a combination of active agent and another compound or composition, inert (for example, a detectable agent, carrier, solid support or label) or active, such as an adjuvant.

A “pharmaceutical composition” is intended to include the combination of an active agent with a carrier, inert or active, making the composition suitable for diagnostic or therapeutic use in vitro, in vivo or ex vivo.

As used herein, the term “pharmaceutically acceptable carrier” encompasses any of the standard pharmaceutical carriers, such as a phosphate buffered saline solution, water, and emulsions, such as an oil/water or water/oil emulsion, and various types of wetting agents. The compositions also can include stabilizers and preservatives. For examples of carriers, stabilizers and adjuvants, see Martin, REMINGTON'S PHARM. SCI, 15th Ed. (Mack Publ. Co., Easton (1975)).

As used herein, the term “inducing an immune response in a subject” is a term well understood in the art and intends that an increase of at least about 2-fold, more preferably at least about 5-fold, more preferably at least about 10-fold, more preferably at least about 100-fold, even more preferably at least about 500-fold, even more preferably at least about 1000-fold or more in an immune response to an antigen (or epitope) can be detected (measured), after introducing the antigen (or epitope) into the subject, relative to the immune response (if any) before introduction of the antigen (or epitope) into the subject. An immune response to an antigen (or epitope), includes, but is not limited to, production of an antigen-specific (or epitope-specific) antibody, and production of an immune cell expressing on its surface a molecule which specifically binds to an antigen (or epitope). Methods of determining whether an immune response to a given antigen (or epitope) has been induced are well known in the art. For example, antigen specific antibody can be detected using any of a variety of immunoassays known in the art, including, but not limited to, ELISA, wherein, for example, binding of an antibody in a sample to an immobilized antigen (or epitope) is detected with a detectably-labeled second antibody (e.g., enzyme-labeled mouse anti-human Ig antibody). Immune effector cells specific for the antigen can be detected any of a variety of assays known to those skilled in the art, including, but not limited to, FACS, or, in the case of CTLs, ⁵¹CR-release assays, or ³H-thymidine uptake assays.

By substantially free of endotoxin is meant that there is less endotoxin per dose of cell fusions than is allowed by the FDA for a biologic, which is a total endotoxin of 5 EU/kg body weight per day.

By substantially free for mycoplasma and microbial contamination is meant as negative readings for the generally accepted tests know to those skilled in the art. For example, mycoplasm contamination is determined by subculturing a cell sample in broth medium and distributed over agar plates on day 1, 3, 7, and 14 at 37° C. with appropriate positive and negative controls. The product sample appearance is compared microscopically, at 100×, to that of the positive and negative control. Additionally, inoculation of an indicator cell culture is incubated for 3 and 5 days and examined at 600× for the presence of mycoplasmas by epifluorescence microscopy using a DNA-binding fluorochrome. The product is considered satisfactory if the agar and/or the broth media procedure and the indicator cell culture procedure show no evidence of mycoplasma contamination.

The sterility test to establish that the product is free of microbial contamination is based on the U.S. Pharmacopedia Direct Transfer Method. This procedure requires that a pre-harvest medium effluent and a pre-concentrated sample be inoculated into a tube containing tryptic soy broth media and fluid thioglycollate media. These tubes are observed periodically for a cloudy appearance (turbidity) for a 14 day incubation. A cloudy appearance on any day in either medium indicate contamination, with a clear appearance (no growth) testing substantially free of contamination.

### EXAMPLES

### Example 1: Clinical Study Design to Access Vaccination of Patients with Acute Myeloid Leukemia with Dendritic Cell Tumor Fusions

First stage: Patients will receive DC/AML fusion vaccination in conjunction with GM-CSF following a chemotherapy induced remission.

Primary objective: To assess the toxicity associated with treating AML patients with DC/AML fusion cells in the post-chemotherapy setting [000100] Secondary objective: To explore immunological response to DC/AML fusion vaccination in patients who have achieved a chemotherapy induced remission.

Second stage: To determine if cellular immunity is induced by treatment with monoclonal antibody PD-1 BLOCKADE and DC/AML fusion cells given in conjunction with GM-CSF following a chemotherapy induced remission.

Primary objective: To assess the toxicity associated with treating AML patients with the combination of DC/AML fusion cells and PD-1 BLOCKADE following a chemotherapy induced remission.

Secondary Objectives 1. To determine if cellular immunity is induced by treatment with monoclonal antibody PD-1 BLOCKADE and DC/AML fusion cells in patients who have obtained a complete remission following induction chemotherapy 2. To correlate levels of circulating activated and regulatory T cells with immunologic response 3. To define anti-tumor effects by determining time to disease progression.

Eligibility Criteria

Participants must meet the following criteria on screening examination to be eligible to participate in the study: 1. Patients with AML at initial diagnosis or at first relapse; 2. Patients must be ≧18 years old; 3.Patients must have ECOG performance status of 0-2; 4. Life expectancy of greater than 9 weeks; 5. Laboratories: Bilirubin≦2.0 mg/dL; AST/ALT<3×ULN; Creatinine≦2.0 mg/dl; 6. The effects of DC/AML fusion cells and PD-1 BLOCKADE on the developing human fetus are unknown. For this reason, women of child-bearing potential and men must agree to use adequate contraception (hormonal or barrier method of birth control; abstinence) prior to study entry and for the duration of study participation. Should a woman become pregnant or suspect she is pregnant while participating in this study, she should inform her treating physician immediately; 7. Ability to understand and the willingness to sign a written informed consent document

Exclusion Criteria

Participants who exhibit any of the following conditions at screening will not be eligible for admission into the study. 1. Patients must not have active or history of clinically significant autoimmune disorders/conditions, defined as requiring systemic therapy, including Type I diabetes. Type II diabetes, vitiligo, stable hypothyroidism, and thyroid disease well controlled with thyroid replacement will not be considered exclusion criteria; 2. Because of compromised cellular immunity, patients who have a known history of HIV will be excluded; 3. Patients must not have significant cardiac disease characterized by symptomatic congestive heart failure, unstable angina pectoris, clinically significant cardiac arrhythmia; 4. Patients must not be pregnant. All premenopausal patients will undergo pregnancy testing. Men will agree to not father a child while on protocol treatment. Men and women will practice effective birth control while receiving protocol treatment; 5. Individuals with a history of a different malignancy are ineligible except for the following circumstances. Individuals with a history of other malignancies are eligible if they have been disease-free for at least 5 years and are deemed by the investigator to be at low risk for recurrence of that malignancy. Individuals with the following cancers are eligible if diagnosed and treated within the past 5 years: cervical cancer in situ, and basal cell or squamous cell carcinoma of the skin.

Eligibility Prior to Cell Collections for Dendritic Cell Generation: Inclusion Criteria

1. Patients must have obtained a complete remission with chemotherapy defined by the absence of circulating blasts, and less than 5% blasts on bone marrow examination following hematopoietic recovery; 2. Resolution of all chemotherapy related grade III-IV toxicity as per CTC criteria 4.0; 3.Laboratories: WBC>2,000/μL Platelets>50,000/uL Bilirubin<2.0 mg/dL Creatinine<2.0 mg/dL AST/ALT<3.0×ULN; 4.For patients with evidence of minimal residual disease prior to vaccination, assessment of minimal residual disease status by cytogenetics or FISH will be followed post vaccination.

Exclusion Criteria:

1. Patients must not have serious intercurrent illness such as infection requiring IV antibiotics, or significant cardiac disease characterized by significant arrhythmia, ischemic coronary disease or congestive heart failure; 2. Patients who, with their treating physician, choose to proceed with an allogeneic transplant at the time of remission will not be vaccinated and will come off study

Eligibility Prior to Post-Chemotherapy Immunotherapy (Before First Vaccine for Cohort 1 and Before First Dose of PD-1 BLOCKADE for Cohort 2)

Resolution of all chemotherapy related grade III-IV toxicity as per CTC criteria 4.0; Laboratories: WBC>2.0×103/uL Platelets>50,000/uL Bilirubin<2.0 mg/dL Creatinine<2.0 mg/dL AST/ALT<3.0×ULN; At least 2 doses of fusion vaccine were produced

### Example 2: Isolation of Tumor Cells

At the time of disease presentation, eligible patients will undergo collection of leukemia cells. Bone marrow aspirates will be obtained (20-30 cc) under local anesthesia and mononuclear cells will be isolated by Ficoll density gradient centrifugation. Patients who have a presenting WBC20K or greater with at least 20% circulating blasts may have tumor cells collected from peripheral blood rather than from a bone marrow collection. In such cases, 50 cc of peripheral blood will be collected and mononuclear cells will be isolated by Ficoll density centrifugation. If leukapheresis is required for clinical indications, the product may be used for subsequent vaccine preparation. Autologous plasma will be obtained by harvesting supernatant following Ficoll centrifugation of 50-100 ml of peripheral blood. Peripheral blood or bone marrow mononuclear cells will be frozen in 10%/DMSO/90% autologous plasma stored in liquid nitrogen. A minimum of 5×10(7) PBMC will be required to go forward with freezing for subsequent vaccine preparation. An aliquot of the tumor cells will undergo immunohistochemical staining and/or FACS analysis for expression of tumor markers (including CD64, CD14, CD38, CD117, CD34, MUC-1), and co-stimulatory molecules. The ability of the leukemia cells to induce proliferation of allogeneic T cells will be measured. A repeat marrow harvest may be performed if the first marrow harvest does not yield adequate tumor cells.

Following collection of tumor cells, leukemia therapy will be administered according to current standards of practice at the discretion of the treating physician. Patients are permitted to receive hydroxyurea prior to initiation of tumor cell collection if clinically indicated. Patients who achieve remission status as defined by absence of circulating blasts, and less than 5% blasts on bone marrow exam following hematopoietic recovery will proceed with immunotherapy. When patients are in remission, leukemic blasts will be thawed, cultured and viability as well as gram stain will be assessed. An aliquot from this sample will undergo microbiological assessment.

When cell yields allow, two doses of 1×10⁵-1×10⁶ cells (based upon cell availability) will be resuspended in PBS, irradiated to 6,000 rads (60 Gy) and frozen at −30° C. for subsequent DTH testing. Remaining cells may be frozen for use in subsequent in vitro assays. Tumor lysate will be prepared by freeze/thawing of an aliquot of tumor cells for immunological analysis.

### Example 3: Chemotherapy

Patients receive 1-2 cycles of induction chemotherapy as per standard of care or on a clinical trial. Patients who receive hypomethylating agents may receive up to 4 cycles of therapy to achieve remission as per standard of care. Choice of induction therapy will be determined at the discretion of the treating physician. Patients not in remission after two cycles of induction chemotherapy will be removed from study. Patients receiving hypomethylating agents who are not in remission after four cycles of chemotherapy will be removed from the study. 0-4 cycles of post-remission chemotherapy may be given. The choice of post-remission therapy and number of cycles will be determined at the discretion of the treating physician. Post-remission therapy should begin from 1 week to 3 months after recovery of peripheral blood counts following induction therapy. Patients will undergo post chemotherapy immunotherapy once that they have recovered from grade 3-4 toxicity. Participants will begin immunotherapy with DC/AML fusion vaccination (cohort 1) or DC/AML fusion vaccination in conjunction with PD-1 BLOCKADE (cohort 2), 4-12 weeks following the last cycle of chemotherapy

### Example 4: Isolation of DC

Patients who achieve complete remission and meet eligibility criteria will undergo leukapheresis to obtain adequate numbers of PBMC. Leukapheresis will be performed when patients are documented to be in complete remission, after recovery of blood counts from the first or second cycle of chemotherapy. When possible, this will be performed via peripheral access. If peripheral access is inadequate, patients will undergo placement of a temporary central venous catheter. After completion of leukapheresis, PBMC will be quantified. If an inadequate yield of PBMC is obtained for the patient's dose requirement, a repeat procedure will be performed.

PBMC will be isolated from the leukapheresis product and cultured in the presence of autologous plasma for 1-2 hours. The adherent fraction will be cultured in the presence of 1% autologous plasma/RPMI medium with 12.5 ng/ml rhIL-4 and 1000 U/ml GM-CSF for five to seven days to generate dendritic cells. 25 ng/ml of TNFα will be then be added for 48-72 hours to enhance DC maturation. Viability and gram stain will be assessed prior to fusion.

These cells will be assessed for morphologic characteristics and expression of characteristic DC markers that include CD11c, HLA DR, CD80, CD86, and CD83. In addition expression of CD117 and MUC-1 will be determined. Functional properties will be assessed using MLR assays in which DC will be co-cultured with allogeneic T cells. T cell proliferation will be measured via tritiated thymidine incorporation

### Example 5: Preparation of DC/AML Fusions

Vaccine preparation may occur upon completion of induction/re-induction therapy, or upon completion of up to 4 cycles of post-remission chemotherapy. Samples will be frozen as outlined below and thawed at the time of vaccine administration. Tumor cells and DC at ratio of 1:10-1:3 (dependent on cell yields) will be mixed and extensively washed in serum-free medium (RPMI1640). After low speed centrifugation, the cell pellets will be re-suspended in 500 μl of 50% solution of polyethylene glycol (PEG) in Dulbecco's phosphate buffered saline without Ca⁺⁺, Mg⁺⁺. After one to five minutes, the PEG will be progressively diluted by the slow addition of serum-free medium. The cells will be washed free of PEG and cultured in RPMI 1640 with 10% autologous plasma and GM-CSF in a 5% CO₂ atmosphere at 37° C. The percentage of the cell population that represent DC/tumor fusions will be determined by quantifying the cells as defined by dual expression of unique DC and leukemia markers such as: a) CD86 and CD117 or CD 34 or MUC-1 orb) CD83 and CD117 or CD34 or MUC-1; c) if the leukemia cells do not express DR, then DR and CD117 or CD34 or MUC-1 as measured by immunocytochemical staining and/or

FACS analysis. Dosing will be determined by the absolute number of fusion cells identified in this manner. Fusion cells must demonstrate >10% fusion efficiency to proceed with vaccination. The fusion cells will then be separated into appropriate aliquots of fusion cells. 2-3 doses of 5×10⁶ fusion cells, depending on cell yields, will be prepared and will be frozen in 10% DMSO/90% autologous plasma in liquid nitrogen. At that time, appropriate aliquots will be sent for microbiological analysis.

### Example 6: Patient Monitoring

Within 14 days of initiating post-remission immunotherapy, patients will undergo:

1—Medical History, Physical Exam, Assessment of Performance Status,

2—Bone Marrow Aspirate and Biopsy and an additional sample of 5-10cc may be collected for immune monitoring studies. For patients with a cytogenetic abnormality at presentation, cytogenetics/FISH will be assessed.*

3—EKG,

4—Laboratory evaluation: CBC with differential, liver function tests (LFTs) (including; ALT, AST, total bilirubin, direct bilirubin**, LDH, Aaline Phosphatase), Electrolytes (Na, K, Cl, CO2, Ca, Mg, PO4) BUN, creatinine erythrocyte sedimentation rate (ESR), and antinuclear antibody (ANA) TSH; Research blood work will be sent for assessment of: DC subsets, immunologic response to tumor lysate (proliferation, IFNγ, and tetramer studies) DTH skin testing to candida and irradiated tumor cells.

5—Assignment to cohort. Patients will be assigned to either cohort 1 (DC/AML fusion vaccine alone) or cohort 2 (DC/AML fusion vaccine+PD-1 BLOCKADE.) The first 25 patients will be assigned to cohort 1. Subsequent patients will be assigned to cohort 2.

*Bone marrow aspirate and biopsy may be done within 30 days of initiation of

Immunotherapy

**Direct bilirubin only required if Total bilirubin is not within normal limits

Evaluation During Immunotherapy Period

The following evaluation will be performed prior to each vaccine and each dose of PD-1 BLOCKADE (+/−2 days):

1—Medical History, Physical Exam, Assessment of Performance Status

2—Vaccine Associated Toxicity Assessment

3—Laboratory evaluations: CBC with differential, liver function tests (LFTs) (including; ALT, AST, total bilirubin, direct bilirubin**, LDH, Alkaline Phosphatase),

electrolytes(Na, K, Cl, CO2, Ca, Mg, PO4), BUN, Creatinine erythrocyte sedimentation rate (ESR)*,antinuclear antibody (ANA)* TSH*ECG*Research blood work will be sent for assessment of:

DC subsets, immunologic response to tumor lysate (proliferation, IFNγ, and tetramer studies)

The following will be performed the weeks that immunotherapy is not asministered +/−2 days:

1—Medical History, Physical Exam, Assessment of Performance Status

2—Treatment Associated Toxicity

3—Laboratory evaluations: CBC with differential, liver function tests (LFTs) (including; ALT, AST, total bilirubin, direct bilirubin**, LDH, Alkaline Phosphatase), electrolytes(Na, K, Cl, CO₂, Ca, Mg, PO4), BUN, Creatinine

**Direct bilirubin only required if Total bilirubin is not within normal limits

Post-Remission Immunotherapy

4-12 weeks following the last cycle of chemotherapy, patients will be reassessed for eligibility for immunotherapy. Patients meeting eligibility criteria outlined in section 5 will begin immunotherapy between 4-12 weeks following chemotherapy. Patients who do not meet eligibility by 12 weeks following the last cycle of chemotherapy will come off study.

### Example 7: Vaccination Schedule

Cohort 1: Patients will be vaccinated with 5×10⁶ fusion cells in the upper thigh region. The site will be alternated for each vaccine administration (right and left extremity). Vaccination will be administered subcutaneously using a 25-gauge ⅝-inch needle. On the day of vaccination, the clinical research nurse/physician assistant will administer 100 ug of GM-CSF subcutaneously at the site of the vaccine. The patient will be trained to inject the remaining three GM-CSF injections (100 ug dose once a day) for self-administration subcutaneously at home. Tumor vaccine will be administered first, followed by GM-CSF injection. If a patient experiences a grade 2 or higher vaccine site reaction during the days when GM-SCF is administered, subsequent doses of GM-CSF may be held until the vaccine site reaction improves to grade 1 or less. For subsequent vaccinations, patients will receive pre-medication with diphenhydramine (Benadryl) 25-50 mg and/or acetaminophen (Tylenol) 650 mg to minimize potential allergic related symptoms. A total of 3 vaccinations will be given at monthly intervals as outlined in the study schema. When cell yields allow for only two doses of vaccine to be generated, patients will receive two doses of vaccine at monthly intervals.

Cohort 2: Patients will receive 3 doses of PD-1 BLOCKADE given at 6 week intervals. Patients will receive acetaminophen 500-1000 mg orally and anti-histamine (for eg. Diphenhydramine 25-50 mg) intravenously 20-60 minutes prior to PD-1 BLOCKADE infusion. The choice of oral antihistamine is at the investigator's discretion. Blood pressure, heart rate, and temperature will be measured after the administration of anti-histamine, and before the initiation of PD-1 BLOCKADE infusion. Vitals signs will be reviewed prior to administration of the study drug. PD-1 BLOCKADE will be infused over approximately 2 hours, in cases where infusion rate is slowed due to an infusion related reaction, the overall infusion time should not exceed 10 hours.

### Example 8: Follow-Up Period

Four weeks following the final vaccination (+/−2 days), patients will undergo:

1—DTH response to Candida,

2—DTH response to irradiated tumor cells,

The following tests are to be performed at the time of the final vaccination and monthly (+/−7 days) for 6 months:

CBC with differential liver function tests (LFTs) (including; ALT, AST, total bilirubin, direct bilirubin*, LDH, Alkaline Phosphatase), electrolytes (Na, K, Cl, CO₂, Ca, Mg, PO4), BUN, creatinine; Direct bilirubin only required if Total bilirubin is not within normal limits

In addition, at one month, three months, and six months (+/−7 days) after their final vaccination patients will undergo:

1—A bone marrow aspirate/biopsy and an additional sample of 5-10 cc may be collected for immune monitoring studies. For patients with a cytogenetic abnormality at presentation, cytogenetics/FISH will be assessed

2—ANA/ESR

3—T-cells subset,

4—Research blood work for the following:

Research blood work will be sent for assessment of:

DC subsets, immunologic response to tumor lysate (proliferation, IFNγ, and tetramer studies) (drawn in 5 green top tubes and sent to Dr. Avigan's at lab BIDMC or Dr. Katz's lab at Rambam Medical Center)

5—TSH

### Example 9: Methodology and Results of Conducted Clinical Study

Methodology of Clinical Study

Patient Characteristics and Study Schema

Patients with newly diagnosed or first relapsed AML were potentially eligible for collection and cryopreservation of primary leukemia samples. Those patients with a history of autoimmune disease, evidence of significant organ dysfunction or other unstable co-morbid medical illness were excluded. Patients achieving complete remission following 1-2 courses of standard induction therapy, who were deemed not appropriate for allogeneic transplantation and were without ongoing grade III-IV chemotherapy related toxicity, were assigned to the vaccine generation and administration phase of the protocol. Vaccines were generated during consolidation chemotherapy with up to 4 cycles of post-remission therapy were permitted at the discretion of the treating physician. The vaccine was administered 1-3 months after the last cycle of chemotherapy. Three doses of 5×106 fusion cells were administered at monthly intervals. No other antileukemia therapy was permitted.

Reagents for Vaccine Characterization and Immunologic Assays

Purified mouse anti-human monoclonal antibodies (mAbs) against HLA-DR, CD80, CD86, CD40, CD83, CD38, CD34, CD117; (ii) CD14 phycoerythrin (PE)-conjugated mouse anti-human mAbs against CD4; (iii) fluorescein isothiocyanate (FITC)-conjugated anti-CD4 (RPA-T4, IgG1), CD8 (RPA-T8, IgG1); (iv) FITC-, PE-conjugated matching isotype IgG1, IgG2a, IgG2b controls; and (v) purified mouse monoclonal IgG1 (MOPC-21) isotype control were purchased from BD PharMingen (San Diego, Calif.). Monoclonal antibody DF3 (anti-MUC1-N) has been described previously 10. Anti-human CD4 TC conjugated, matching isotype control (IgG2a), PE-conjugated anti-human mAbs against IFN-γ (mouse IgG1-B27) and PE-conjugated matching isotype controls (rat IgG1-PE and mouse IgG1-PE) were purchased from Invitrogen (Carlsbad, Calif.). FITC-conjugated goat anti-mouse (IgG1) was purchased from Chemicon International (Temecula, Calif.).

Vaccine Generation, Characterization, and Administration

AML cells were collected via aspiration of 20-30 cc of bone marrow, 40-50 cc of peripheral blood in those patients with high levels of circulating blasts, or leukapheresis collections in patients requiring emergent cytoreduction due to risk of leukostasis. Mononuclear cells were isolated by Ficoll density gradient centrifugation and cryopreserved in 10% DMSO/90% autologous plasma. Patients eligible for vaccine generation underwent a single leukapheresis collection for DC generation and vaccine production. Adherent cells were cultured with 1000 U/mL GM-CSF (Sanofi) and 500 IU/ml IL-4 (Cellgenix USA, Antioch, Ill.) for 5-7 days and matured in the presence of 25 ng/ml TNFα (Cellgenix) for 2-3 days. The DC and thawed autologous AML preparations were analyzed by immunocytochemical staining and then cocultured with polyethylene glycol (PEG) to generate DC/tumor fusions as previously described7. DC/AML fusions were (i) quantified by determining the percentage of cells that co-express unique DC (CD80, CD83 or CD86) and tumor-associated antigens (CD34, CD38, CD64, CD117, or MUC1) by immunohistochemical analysis, (ii) assessed for sterility, (iii) cryopreserved in autologous plasma (90%) and DMSO (10%) in single dose vials of 5×106 fusion cells, and (iv) stored frozen in the vapor phase of liquid nitrogen. At the time of administration, the fused cells were irradiated with 30 Gy and administered as a subcutaneous injection in the upper thigh at 4-week intervals for a total of 3 doses. GMCSF (100 μg) was administered at the vaccine site on the day of vaccination and for 3 days thereafter. As a measure of their potency as antigen presenting cells, the capacity of DC/AML fusion cells to stimulate allogeneic T cell proliferation was assessed. 1×105 T cells obtained from leukopak collections were co-cultured with either DC/AML fusion cells, DCs, or AML blasts at a ratio of 10:1 for 5 days. T cell proliferation was determined by measuring incorporation of [3H] thymidine following overnight pulsing (1 μCi/well) of triplicate samples.

Vaccine Induction of Immunologic Response

Patients underwent serial assessment for levels of circulating and bone marrow derived T cells reactive with whole autologous AML cells and previously identified leukemia associated antigens. Cryopreserved PBMC were collected prior to each vaccination and at 1, 3 and 6 months thereafter. Following the last vaccination, PBMC samples were thawed and 1×106 cells were cultured with lysate generated by repeated freeze thaw cycles of 1×105 autologous leukemia cells for 5 days. Cells were re-stimulated with autologous tumor lysate for 6 hours and cultured overnight with 1 μg/ml GolgiStop. Intracellular expression of IFNγ by CD4+ or CD8+ T cells was determined by FACS analysis of permeabilized cells. Leukemia reactive T cells were also quantified in the bone marrow prior to and 1 month following completion of vaccination in a subset of patients. In HLA-A2.1 patients, the number of circulating CD8+ T cells binding the MUC1, WT1, PR1 pentamers were determined by bidimensional FACS analysis using CD8-FITC and the corresponding pentamer-PE antibody.

Levels of regulatory T cells were quantified by determining the expression of FOXP3 by CD4/CD25 cells using intracellular FACS analysis. PD-1 expression on circulating CD4 and CD8 T cell populations was assessed by flow cytometry. In a subset of patients, vaccine site reactions underwent biopsy and immunocytochemical staining to assess infiltration of CD4, CD8 T cells. Recruitment of native DCs was assessed by CD1a expression in the vaccine site.

Clinical Disease Assessment

Disease assessment was monitored by complete blood counts with differential and a bone marrow aspiration and biopsy within 1 week prior to initiation of vaccination and 4 weeks, three months and six months after completion of therapy. Cytogenetic evaluation was performed on bone marrow specimens in patients who had abnormal cytogenetics at presentation. Patients were monitored for disease relapse with assessments every 3 months.

Statistical Methods

For analysis of immune response to vaccination, fold change of IFNγ by CD4+ or CD8+ T cells between pre vaccine measurement and 1 month after the last vaccination was summarized as median, mean and standard deviation. A paired t test was used to assess whether the ratio of expression of these markers differ from 1. Progression-free survival (PFS), is defined as the time from the date of first vaccine to the date of relapse or death from any cause, and overall survival (OS), defined as the date from first vaccine to the date of death. Kaplan-Meier method was used to summarize the 2 year PFS rate. Greenwood formula was used to estimate standard error for calculating 95% CI. Statistical analysis was performed using SAS/STAT software, Version 9.4 of the SAS System for Windows.

Results of Clinical Study

Patient Characteristics

Characteristics of the 16 evaluable AML patients are summarized in Table 1. Median age was 60 years. Remission induction therapy consisted of 7+3 in 13 patients, 7+3 followed by 5+2 re-induction in 1 patient, MEC in 1 patient, and decitabine in 1 patient. Five patients were characterized as poor risk, 9 intermediate risk and 2 good risk by cytogenetic analysis. The good risk category included 1 patient with an inversion 16 abnormality accompanied by other cytogenetic abnormalities. Four patients completed vaccine generation, but did not receive 2 vaccinations due to relapsed AML (n=3) or ongoing chemotherapy related toxicity precluding initiation of vaccination (n=1). Twenty-eight patients underwent tumor cryopreservation but did not complete vaccine generation (Table 2).

Vaccine Characterization

The mean yield of the DC and AML preparations was 171 and 87×10⁶ cells, respectively. The mean fusion efficiency as determined by the percentage of cells that co-expressed unique DC (CD80, CD86, and CD83) and AML (CD38, CD34, CD117, CD64, or MUC1) antigens was 43%. The mean viability of the DC, AML, and fusion preparations was 91%, 91%, and 86%, respectively. In contrast to AML blasts, the DC and fusion preparations potently stimulated allogeneic T cell proliferation (mean stimulation indices 3.6, 15.7, and 10.9, respectively). 14/16 patients received 3 doses while 2 patients received 2 doses, 1 due to limitations of cell yields and 1 due to disease relapse prior to the third vaccine.

Adverse Events

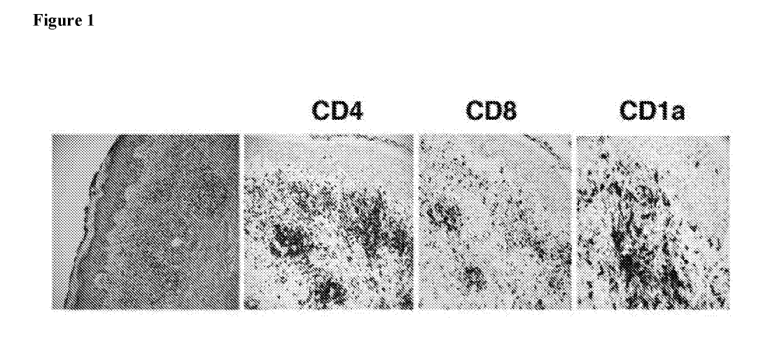

Vaccination was well tolerated and not associated with clinically significant autoimmunity. Possibly related adverse events were transient and of grade 1-2 intensity (Table 3). The most common adverse event was erythema, pruritis and/or induration at the vaccine site. Biopsy of vaccine site reactions demonstrated a dense infiltrate of CD4 and CD8 T cells consistent with recruitment of reactive T cell populations to the vaccine bed (FIG. 1).

Cellular Immunologic Response to Vaccination

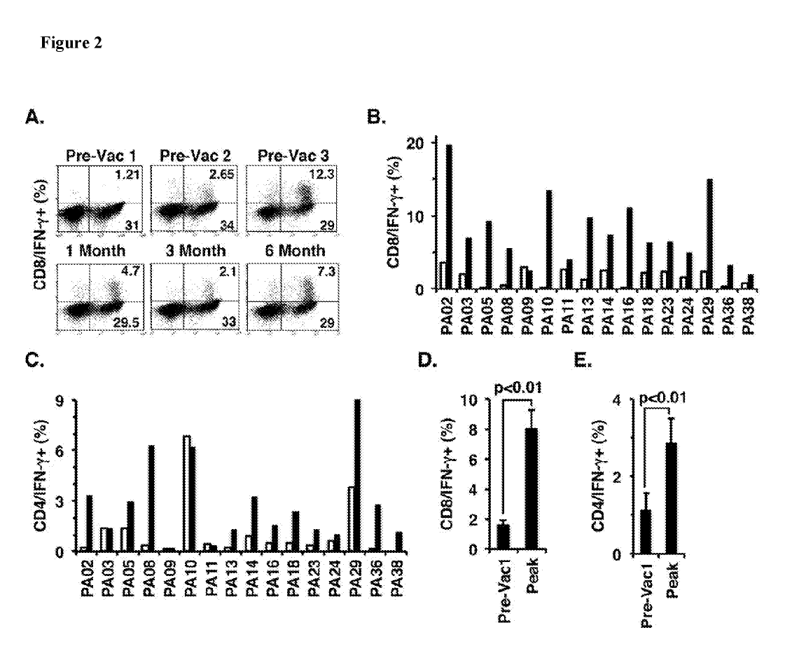

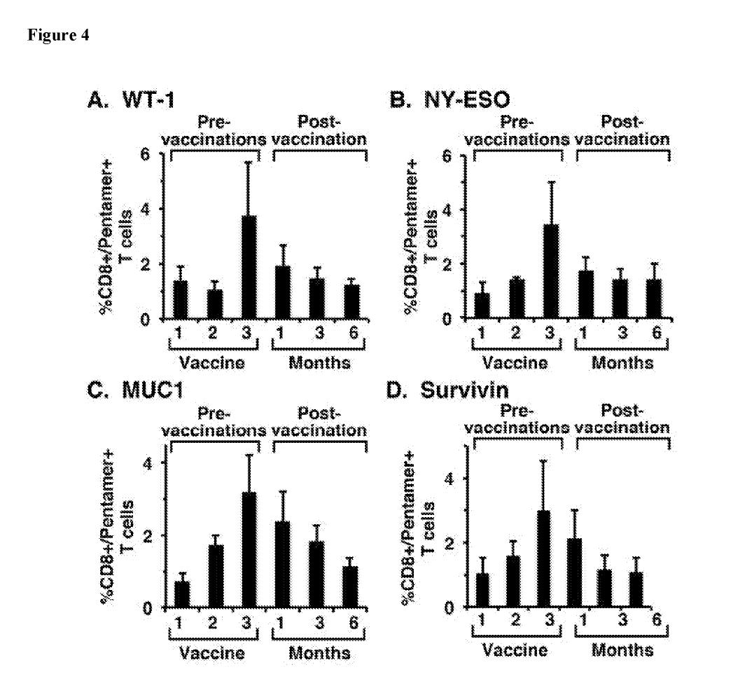

Vaccination with DC/AML fusions induced the expansion of leukemia specific T cells in the peripheral blood and bone marrow as determined by the percentage of CD4 and CD8 T cells expressing IFNy upon ex vivo exposure to autologous tumor lysate. Consistent with these findings, vaccination also resulted in the significant expansion of CD8 T cells recognizing previously identified AML antigens quantified by pentamer analysis in HLAA2.1 patients. Following recovery from consolidation chemotherapy, levels of AML specific T cells were nearly undetectable in the peripheral blood and bone marrow (FIG. 2). Vaccination resulted in a 5.3 and 13.3 fold increase in AML specific CD4 and CD8 T cells comparing pre-vaccination levels to those measured one month following the last vaccine (n=16, p<0.05) (FIG. 2A-E). Circulating leukemia reactive T cells remained elevated 6 months following last vaccination at time of last assessment. Similarly, vaccination was associated an 6.7 fold increase in leukemic reactive CD8+ T cells present in the bone marrow from pre-vaccination to 1 month following the last vaccination (n=5). In HLA-A*0201 patients, vaccination resulted in the expansion of peripheral blood T cells recognizing MUC1 (4.4 fold increase), WT1 (2.7 fold increase), and NY-ESO (3.8 fold increase) tumor antigens (N=4, FIG. 4).

Quantification of Tregs and PD-1 Expressing T Cells in the Peripheral Blood

Preclinical studies have demonstrated that, in addition to activated T cells, vaccination may result in the expansion of inhibitory cell populations that can mute clinical effects. Circulating regulatory T cell populations, defined as co-expressing CD4/CD25 and FOXP3, were quantified prior to each vaccination and serially following vaccination. Circulating regulatory T cells were present at low levels prior to vaccination (mean 7.18%, n=13), and did not increase throughout the period of vaccination (mean 5.57% and 5.43% prior to the second and third vaccine respectively, n=13) or follow up (mean 7.04% at 6 month follow up, n=13). Similarly, PD-1 expression on circulating CD4 and CD8 T cell populations remained unchanged during the period of vaccination and follow up (data not shown).

Clinical Response

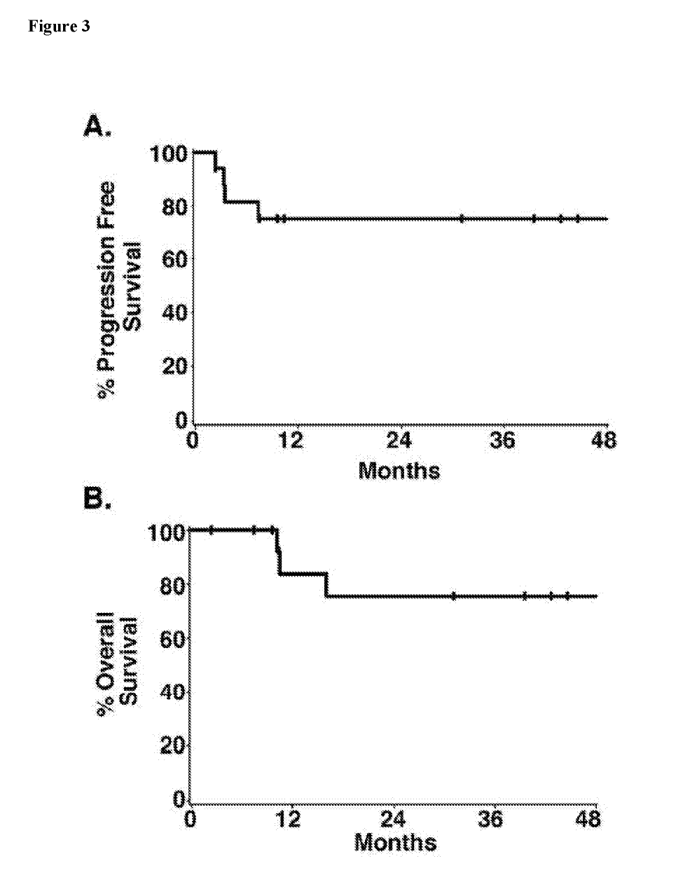

Despite a median age of 60 and 14/16 patients in the study population characterized as intermediate or high-risk disease by cytogenetics, 12 of 16 patients remain in remission (75%) with a median follow up of 43 months. As a notable example, a 77 year old female who relapsed within one year of primary induction chemotherapy underwent vaccination after achieving a second remission. She remains in remission with 53 months of follow up from vaccination. No patient has relapsed later than 1 year from completing chemotherapy. 2 year progression free survival rate is 76% (95% CI 42%, 91%). Median follow up is 43 months. Median PFS and OS have not been reached (FIG. 3).

Clinical Study Observations/Discussion

Examples presented herein demonstrate the striking efficacy of this vaccine in AML patients who achieve a remission following chemotherapy. Twelve of 16 patients (75%) remain in remission with a median of nearly 4 years of follow up and no patients have relapsed after 1 year following chemotherapy. Patients remaining in remission include several over age 70. Remarkably, one patient who relapsed within 1 year of initial chemotherapy underwent vaccination after achieving a second chemotherapy-induced remission and remains in remission over 4 years after completing chemotherapy. The long term remission observed following chemotherapy and vaccination of a patient who had experienced early relapse following initial induction therapy is distinctly unusual in the absence of allogeneic transplantation.

The clinical effects of the vaccine were observed in the context of sustained induction of AML specific immunity as measured by the expansion of AML specific CD4 and CD8 T cells in the peripheral blood. A concomitant rise in leukemia specific T cells in the bone marrow further supports the notion that the immune response is generated in this critical microenvironment. The specificity of the immune response is further supported by the expansion of T cells recognizing leukemia associated antigens, WT1, MUC1, and NYESO. In this context, MUC1 is uniquely expressed by leukemia stem cells, as compared to normal hematopoietic stem cells, consistent with the potential targeting of this self-renewing population. The expansion of AML specific T cells peaked at 2 months after vaccination and persisted at 6 months following treatment, the last time point measured. Thus the role of booster vaccination in maintaining response should be evaluated.

Vaccination with DC/AML fusions was well tolerated with toxicity predominantly limited to reactions associated with recruitment of activated T cells into the vaccine site. This response stands in contrast to toxicities observed with nonspecific activation of the immune system in the setting of negative checkpoint inhibitors and the infusion of constitutively activated T cells, including pneumonitis and the cytokine release syndrome, respectively.

One potential concern is that patient selection involving those patients who underwent vaccination was responsible for the outcomes observed in the trial. Only those patients who achieved remission were eligible for vaccination; however, there was no subsequent selection process that favorably biased the population. Vaccination was restricted to patients not judged to be appropriate for allogeneic transplantation due to age, comorbidities, or absence of a donor.

TABLE 3
Reasons for Study Participant Withdrawal

| | # of |
|---|---|
| Reason for Withdrawal | Patients |
| Allogeneic Transplant in Remission | 15 |
| Primary Induction Failure | 7 |
| Died during remission induction therapy | 3 |
| Patient Choice | 3 |
| Insufficient Tumor | 1 |

### OTHER EMBODIMENTS

While the invention has been described in conjunction with the detailed description thereof, the foregoing description is intended to illustrate and not limit the scope of the invention, which is defined by the scope of the appended claims. Other aspects, advantages, and modifications are within the scope of the following claims.

We claim:

1. A method of treating acute myeloid leukemia (AML) cancer in a patient comprising administering to said patient in post chemotherapy induced remission or active disease a composition comprising a population of autologous dendritic cell/AML tumor cell fusions (DC/AML fusions).

2. The method of claim 1, wherein the composition is administered 4 to 12 weeks following the completion of chemotherapy.

3. The method of claim 1, wherein the composition comprises about 1×10⁶ to 1×10⁷ DC/AML cell fusions.

4. The method of claim 3, wherein in the composition comprises about 5×10⁶ DC/AML cell fusions

5. The method of claim 1, wherein the composition is administered at 4 week intervals.

6. The method of claim 1, further comprising administering GM-CSF.

7. The method of claim 6, wherein said GMCSF is administered daily for 4 days.

8. The method of claim 6, wherein the GM-CSF is administered at a dose of 100 ug.

9. The method of claim 5, comprising further administering GM-CSF at each dose of said DC/AML cell fusions.

10. The method of claim 1, further comprising administering said subject a checkpoint inhibitor.

11. The method of claim 10, wherein the checkpoint inhibitor is administered one week after the DC/AML fusions.

12. The method of claim 10, wherein the checkpoint inhibitor is a PD1, PDL1, PDL2, TIM3, LAG3 inhibitor.

13. The method of claim 12, wherein the checkpoint inhibitor is a PD1, PDL1, TIM3, LAG3 antibody.

14. The method of claim 10, wherein the checkpoint inhibitor is administered at 6 week intervals.

15. The method of claim 1, wherein the further comprising administering an agents that target regulatory T cells

16. The method of claim 1, further comprising administering said subject an immunomodulatory agent.

17. The method of claim 16, where the immunomodulatory agent is lenalidomide, pomalinomide or apremilast.

18. The method of claim 1, further comprising administering said subject a TLR agonist, CPG ODN, polyIC, or tetanus toxoid.

19. A method of producing a fused cell population, comprising:
a. providing a population of AML cells and a population of dendritic cells (DC) obtained from the same subject;
b. mixing the tumor cells and the DC at a ratio of 1:10 to 1:3 to produce a mixed cell population;
c. adding polyethylene glycol (PEG) to the mixed cell population in an amount sufficient to mediate fusion of the tumor cell and DC cell to produce a fused cell population.

20. The method of claim 19, further comprising:
d. culturing the fused cell population in a culture media with 10% heat inactivated autologous plasma and GM-CSF.

21. The method of claim 19, further comprising
e. quantifying the number of cells in the fused cell population that co-express unique DC or leukemia markers.

22. The cell produced by the method of claim 19.

23. The cell population of claim 22, wherein the cell population is substantially free of endotoxin, microbial contamination and mycoplasma.
